(19) United States
(12) Patent Application Publication
(10) Pub. No.: US 2020/0341004 A1
(43) Pub. Date: Oct. 29, 2020

(54) MASS SPECTROMETRY METHODS FOR CARCINOMA ASSESSMENTS

(71) Applicant: Frontier Diagnostics, LLC, Smyrna, TN (US)

(72) Inventors: Jeremy L. Norris, Smyrna, TN (US); Richard M. Caprioli, Brentwood, TN (US); Jason B. Robbins, Nashville, TN (US); Jessica Lindsey Moore, Nashville, TN (US); Nathan Heath Patterson, Nashville, TN (US)

(21) Appl. No.: 16/759,686

(22) PCT Filed: Oct. 29, 2018
(86) PCT No.: PCT/US18/58013

§ 371 Date: Apr. 27, 2020

Related U.S. Application Data
(60) Provisional application No. 62/578,214, filed on Oct. 27, 2017.

(51) Int. Cl. G01N 33/68; G01N 30/72; H01J 49/04; H01J 49/16

(52) U.S. Cl. CPC G01N 33/6851; G01N 30/72; H01J 49/40; H01J 49/167; G01N 2800/20; H01J 49/0418

(57) ABSTRACT

The present invention is directed to a mass spectrometry approach to identifying carcinomas or tissue abnormalities, and distinguishing carcinomas or tissue abnormalities.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The present invention is directed to methods for identifying and differentiating squamous cell carcinomas, basal cell carcinomas, verrucas, and seborrheic keratosis using MALDI imaging mass spectrometry methods. The present invention is further directed to methods for identifying and differentiating manifestations of autoimmune disorders (such as psoriasis, rheumatoid arthritis, and the like) from cancers associated with tissues where such autoimmune disorders materialize, via using MALDI imaging mass spectrometry methods.

### BACKGROUND OF THE INVENTION

Carcinoma is a type of cancer that arises from cells that comprise the skin or the tissue lining organs, such as the liver or kidneys. Some common types of carcinoma include, but are not limited to, basal cell carcinoma, squamous cell carcinoma, renal cell carcinoma, ductal carcinoma in situ, invasive ductal carcinoma, and adenocarcinoma.

An autoimmune disorder is a condition wherein an immune response is mounted against the subject's own cells resulting in the subject's immune system attacking its very own tissue. Non-limiting examples of an autoimmune disorder include psoriasis, psoriatic arthritis, Crohn's disease, rheumatoid arthritis.

### SUMMARY OF THE INVENTION

An aspect of the invention is directed to methods of distinguishing a squamous lesion. In one embodiment, the method comprises subjecting a sample from a subject to mass spectrometry; obtaining a mass spectrometric profile from said sample; comparing the sample mass spectrometric profile to a profile obtained from a known normal sample, a tissue abnormality sample, and/or carcinoma sample; and identifying the lesion as a carcinoma or tissue abnormality based on the comparison between the mass spectrometric profile and the known profile or profiles. In one embodiment, the sample is a skin lesion sample or gastrointestinal lesion sample. In another embodiment, the tissue abnormality is Seborrheic Keratosis or Verruca Vulgaris. In a further embodiment, the carcinoma is basal cell carcinoma, squamous cell carcinoma, renal cell carcinoma, ductal carcinoma in situ, invasive ductal carcinoma, or adenocarcinoma.

An aspect of the invention is directed to methods of identifying carcinoma or a tissue abnormality. In one embodiment, the method comprises subjecting a sample from a subject to mass spectrometry; obtaining a mass spectrometric profile from the sample; comparing the sample mass spectrometric profile to a profile obtained from a known normal, a tissue abnormality, and/or carcinoma sample; and identifying said lesion as a carcinoma or tissue abnormality based on the comparison between said mass spectrometric profile and said known profile or profiles. In one embodiment, the sample is a skin lesion sample or gastrointestinal lesion sample. In another embodiment, the tissue abnormality is Seborrheic Keratosis or Verruca Vulgaris. In a further embodiment, the carcinoma is basal cell carcinoma, squamous cell carcinoma, renal cell carcinoma, ductal carcinoma in situ, invasive ductal carcinoma, or adenocarcinoma.

Another aspect of the invention is directed to at least one biomarker for the identification of carcinoma, T-cell lymphoma, tissue abnormalities, or a combination thereof in a tissue sample from a subject. In embodiments, the biomarker comprises a molecular signature obtained via mass spectrometry. The molecular signature can comprise one or more m/z peaks that are selectively present in carcinoma tissue. The molecular signature can comprise one or more peaks that are selectively present in any of various tissue abnormalities including, but not limited to Seborrheic Keratosis, Verruca Vulgaris, psoriasis, psoriatic arthritis, Crohn's disease, rheumatoid arthritis, or a combination thereof

An additional aspect of the invention is directed to a diagnostic kit for identifying a tissue as normal, a carcinoma, T-cell lymphoma, a tissue abnormality, or a combination thereof. In embodiments, the kit includes the biomarkers listed or otherwise referenced herein and a means for measuring one or a combination of molecular profiles in a tissue sample. In an embodiment, the means for measuring one or a combination of molecular profiles comprises mass spectrometry.

Other objects and advantages of this invention will become readily apparent from the ensuing description.

### BRIEF DESCRIPTION OF THE FIGURES

FIG. 1 shows a photograph of a hematoxylin and eosin (H&E)-stained cutaneous squamous lesion.

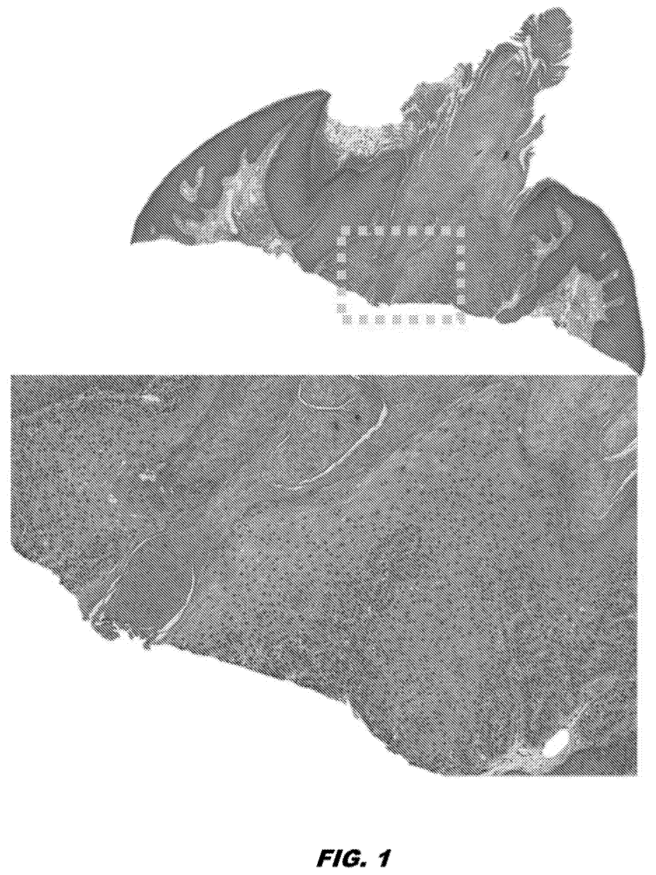

FIG. 2 shows imaging mass spectrometry. The diagnostic platform built around imaging mass spectrometry consists of 3 components: 1) a novel collaborative web interface that controls 2) a mass spectrometer, and 3) a data analysis pipeline for classification of the histology-directed mass spectral data.

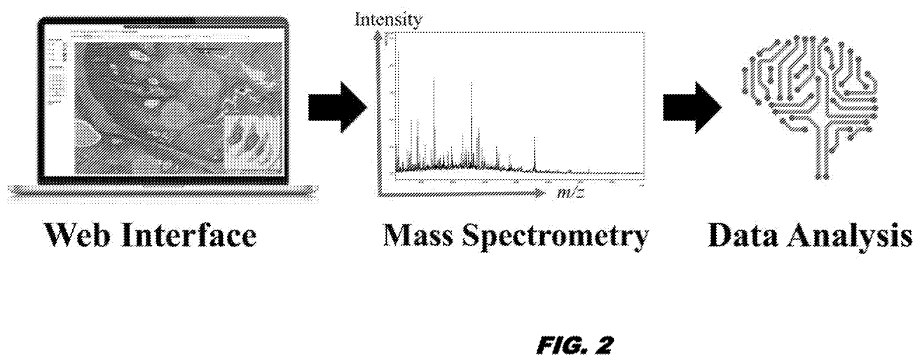

FIG. 3 depicts the applications that the imaging mass spectrometry platform can be used with respect to gastrointestinal disorders.

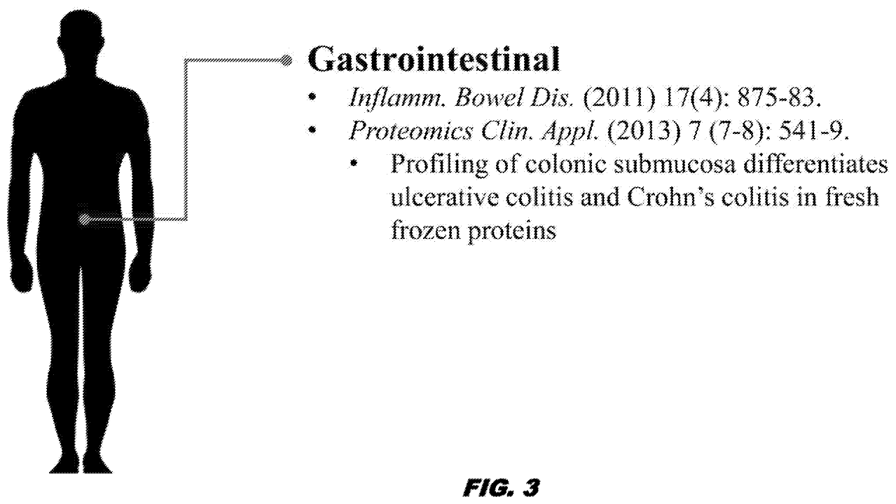

FIG. 4 depicts the applications that the imaging mass spectrometry platform can be used with respect to bone/muscle disorders.

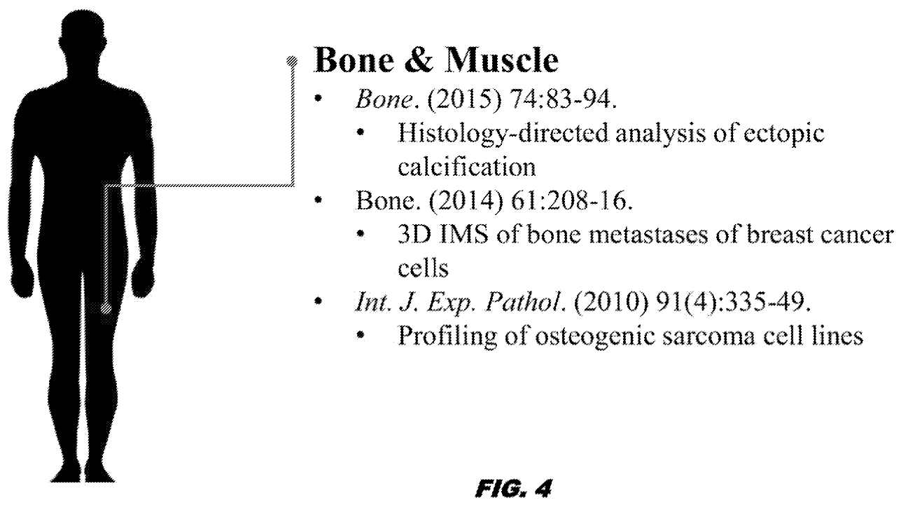

FIG. 5 depicts the applications that the imaging mass spectrometry platform can be used with respect to skin disorders/diseases and wound healing.

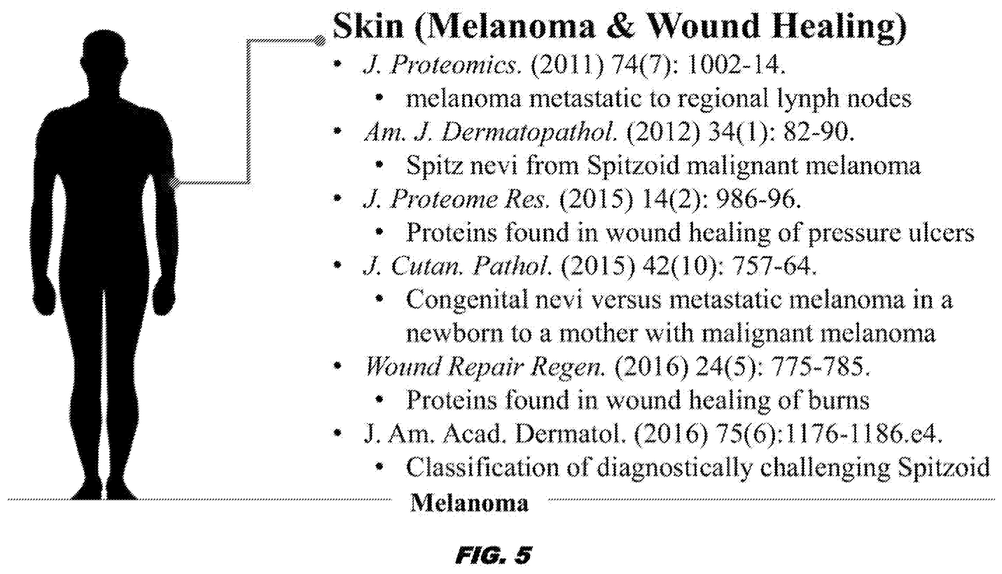

FIG. 6 is a diagram of the histology-directed MALDI Mass spectrometry process.

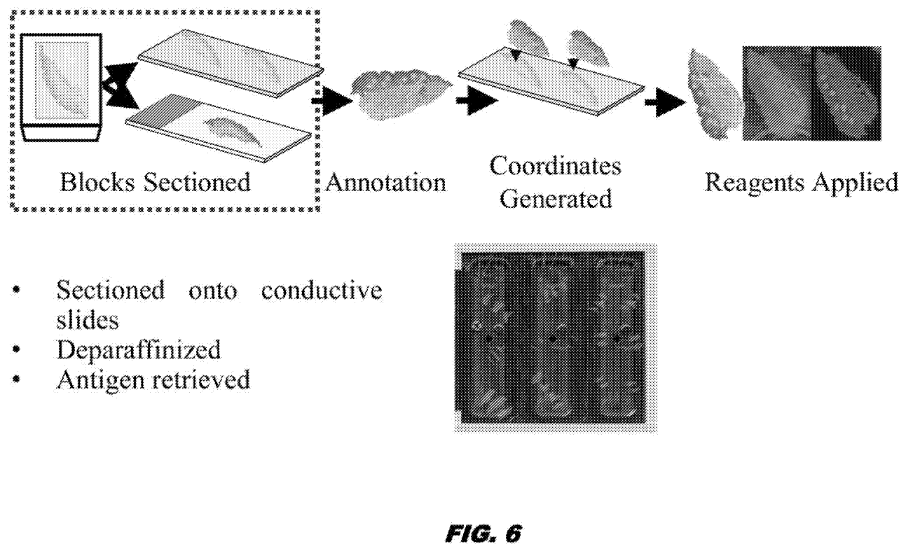

FIG. 7 is a schematic of the histology-directed MALDI Mass spectrometry process.

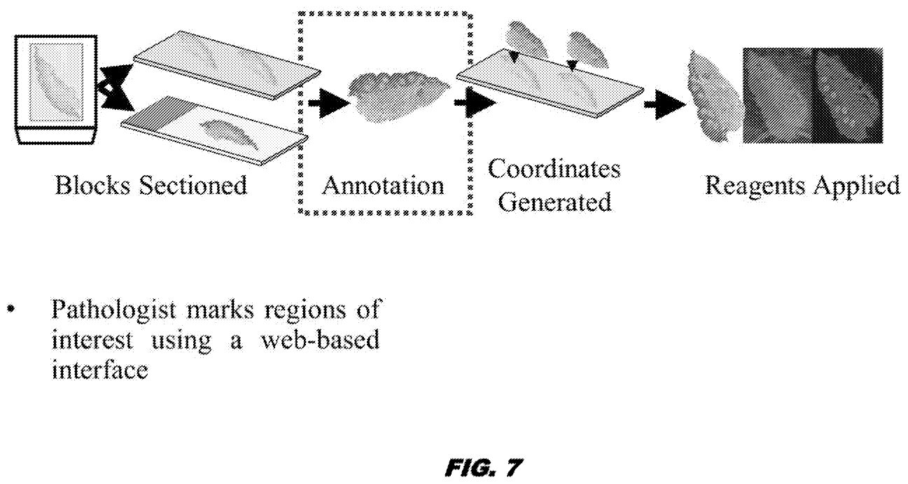

FIG. 8 shows the Pathology Interface for Mass Spectrometry (PIMS). Web based interface allows pathologists to guide mass spectrometry-based assays and remotely collaborate.

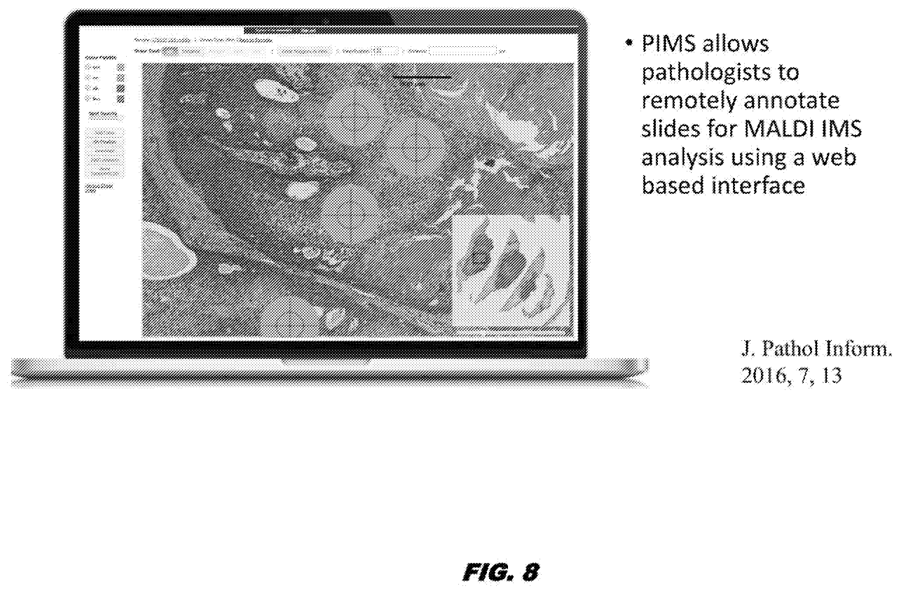

FIG. 9 shows the Pathology Interface for Mass Spectrometry (PIMS).

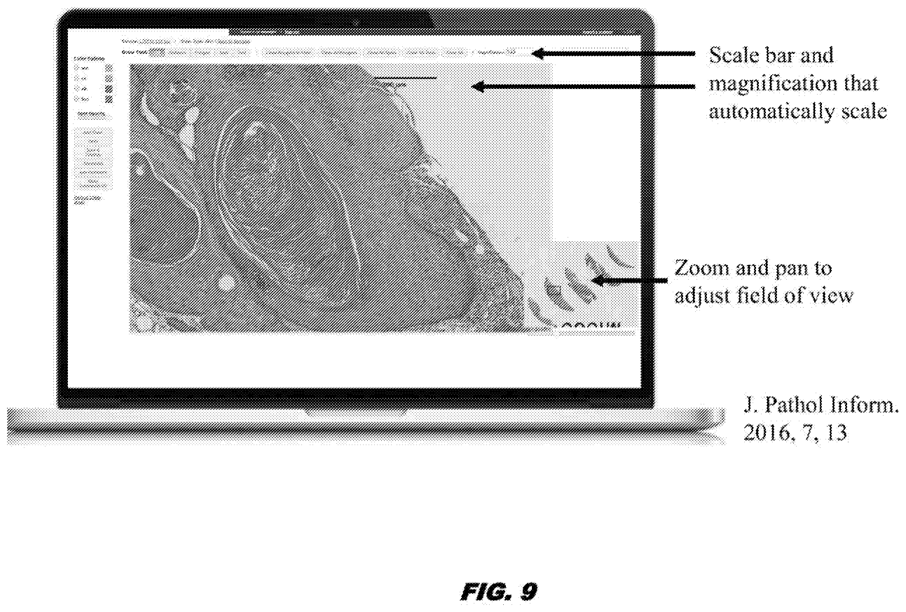

FIG. 10 shows the Pathology Interface for Mass Spectrometry (PIMS).

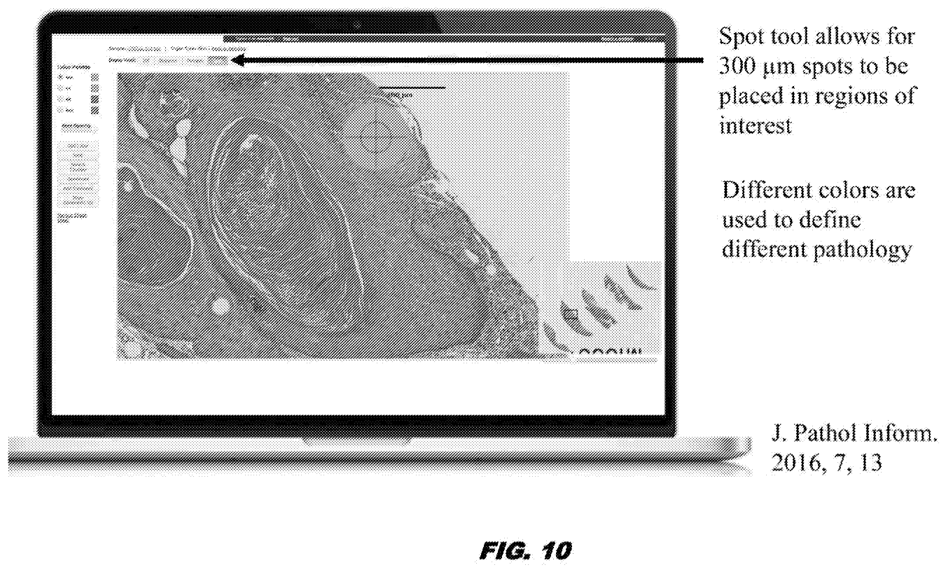

FIG. 11 shows the Pathology Interface for Mass Spectrometry (PIMS).

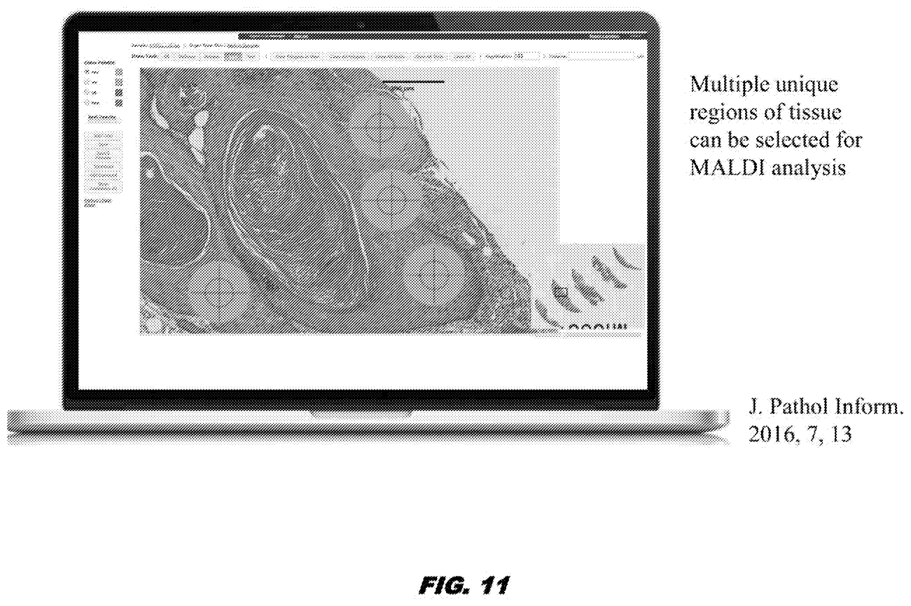

FIG. 12 is a diagram of the histology-directed MALDI Mass spectrometry process.

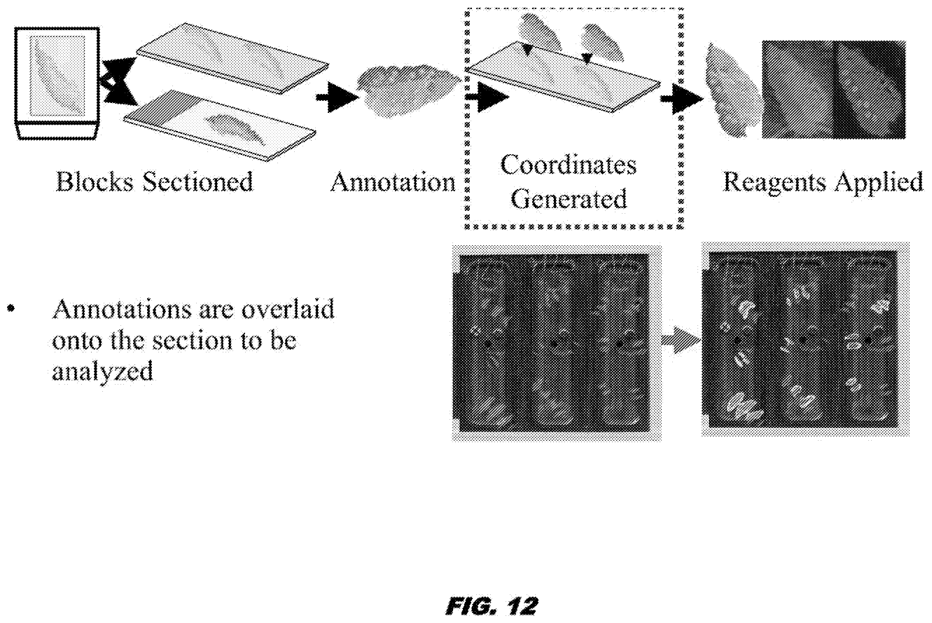

FIG. 13 is a schematic of the histology-directed MALDI Mass spectrometry process.

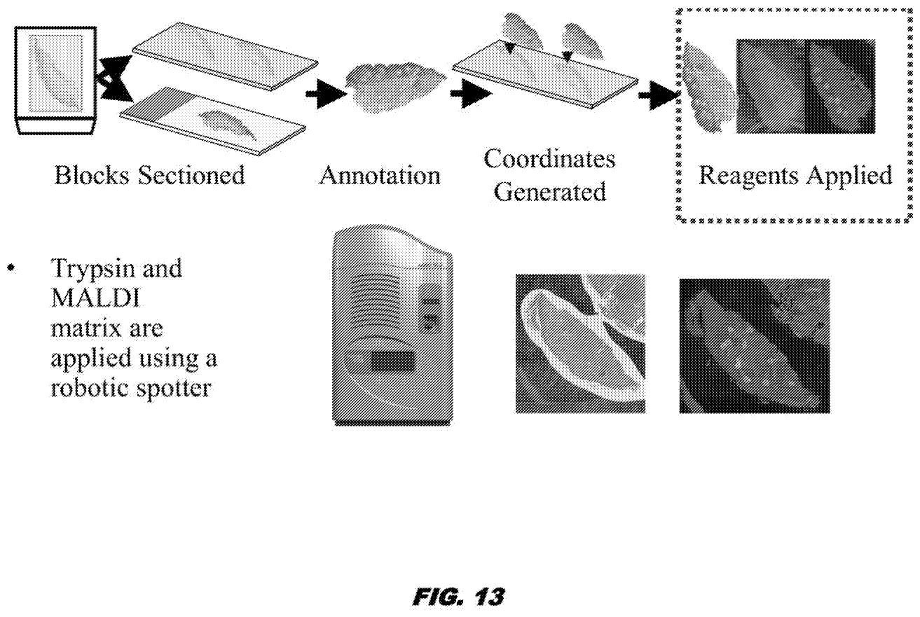

FIG. 14 is a diagram of the histology-directed MALDI Mass spectrometry process.

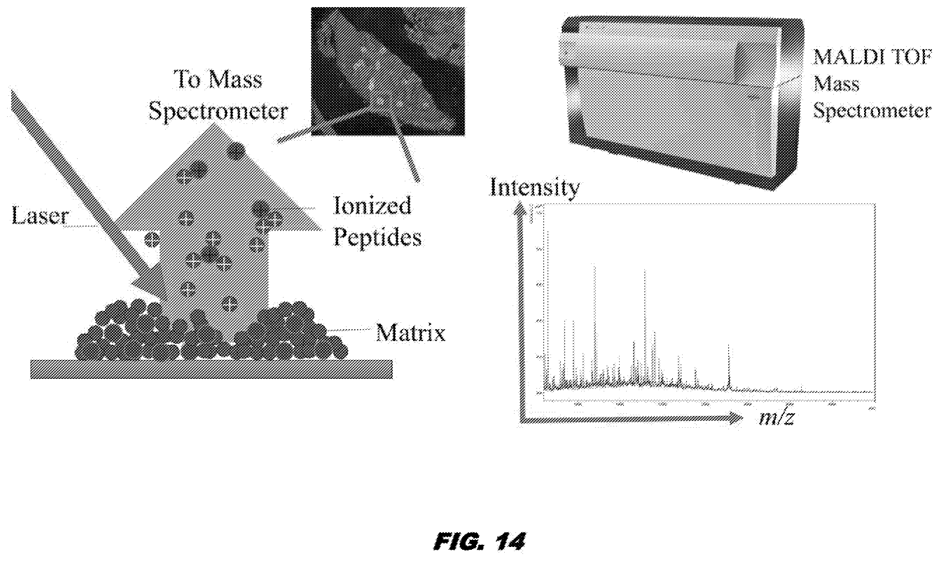

FIG. 15 is a diagram of the general data analysis workflow for the histology-directed MALDI Mass spectrometry process. After the spatially targeted MALDI-MS data is acquired, machine learning algorithms are applied to mathematically model the important class-wise variation. Once these models are constructed from well-characterized training data, they can be applied to previously unknown data and classify it into one of the original classes. Importantly, while model building can sometimes be time consuming for very large data sets (hours to days), classification generally can be done in less than a second, delivering very rapid results after data acquisition.

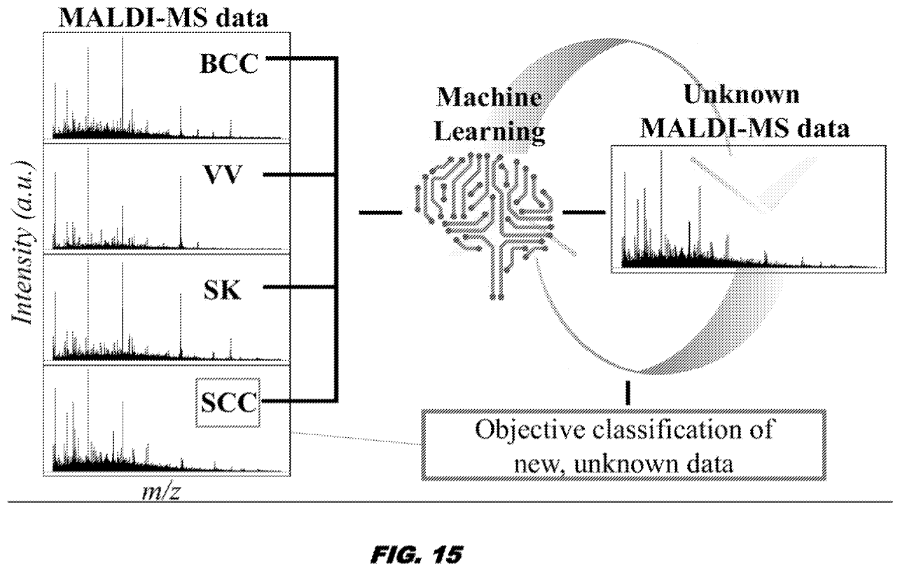

FIG. 16 shows a study design with data obtained from 130 patient samples. BCC: Basal cell carcinoma; SCC: squamous cell carcinoma; SK: Seborrheic Keratosis; VV: Verruca Vulgaris.

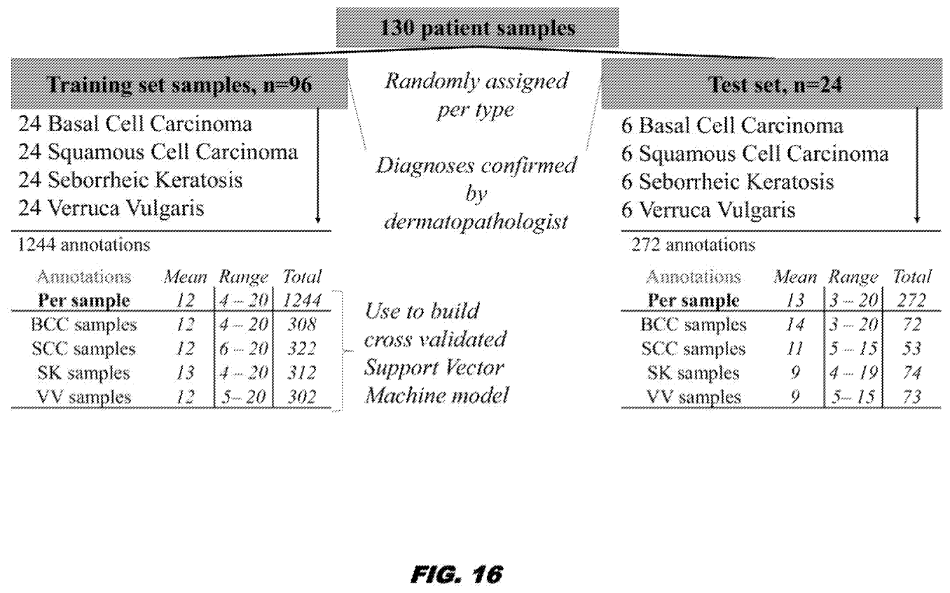

FIG. 17 shows a study design with data obtained from 130 patient samples. BCC: Basal cell carcinoma; SCC: squamous cell carcinoma; SK: Seborrheic Keratosis; VV: Verruca Vulgaris.

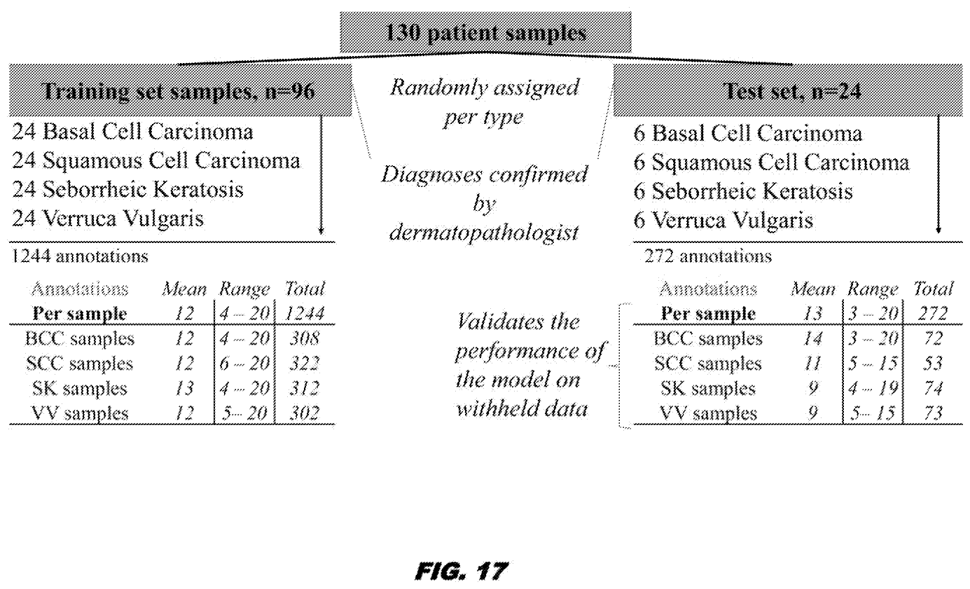

FIG. 18 shows test set results as a majority per patient.

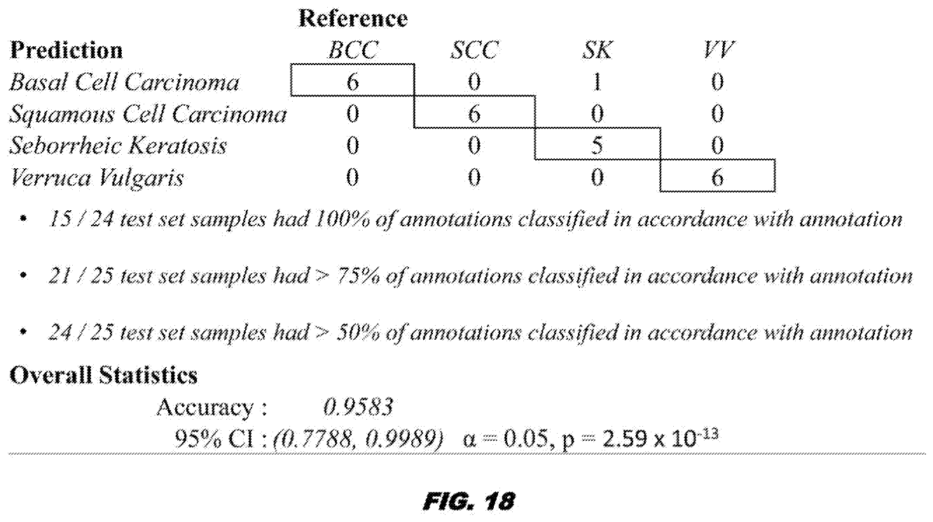

FIG. 19 shows test set results as mass spectra classification.

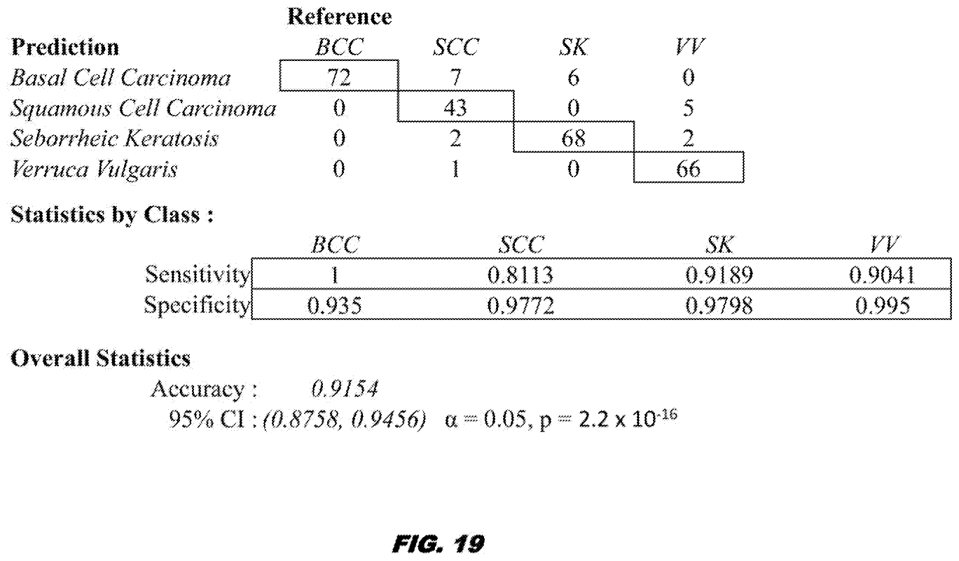

FIG. 20 shows H&E sections for Verruca Vulgaris (left panels), Seborrheic Keratosis (middle panels), and squamous cell carcinoma (right panels). Basal cell carcinoma is noted in blue while squamous cell carcinoma is noted in yellow. Each spot is 300 μm.

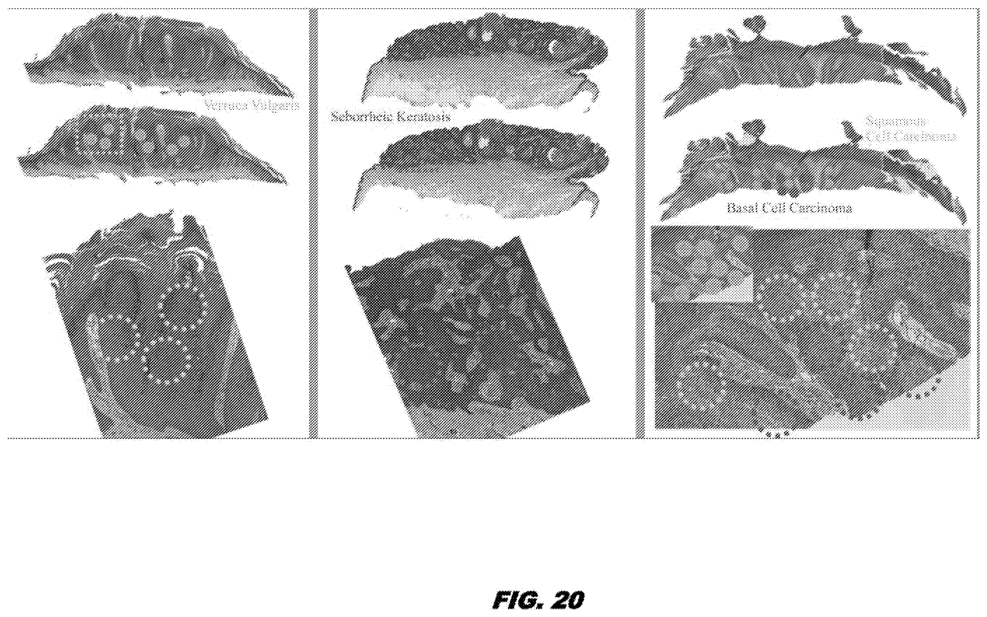

FIG. 21 shows H&E sections for Seborrheic Keratosis classified as basal cell carcinoma. Each spot is 300 μm.

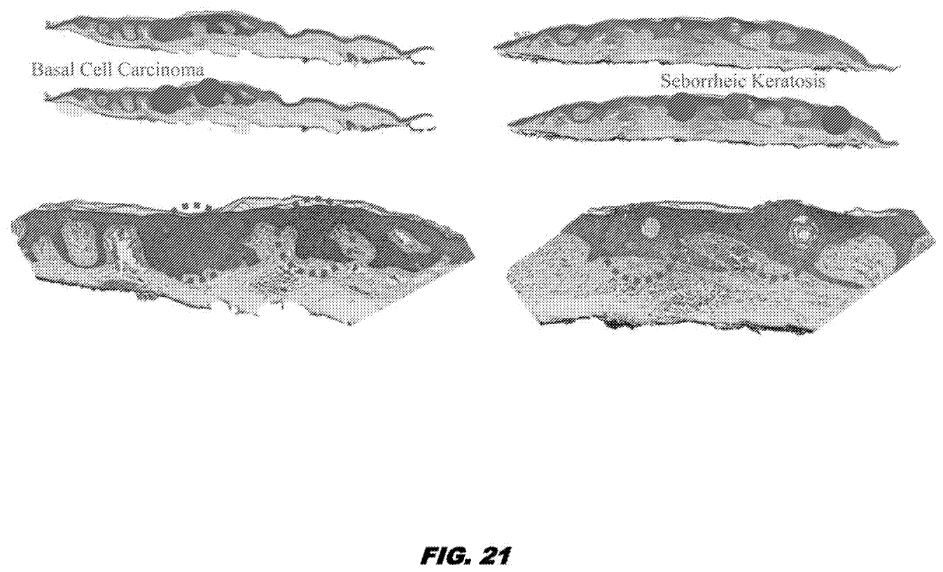

FIG. 22 shows light microscopy and fluorescent microscopy images.

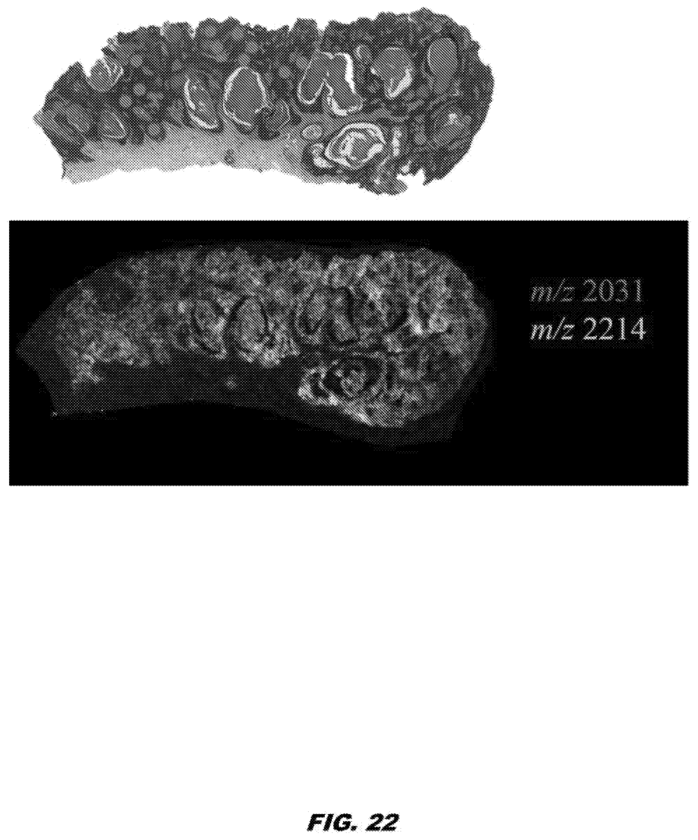

FIG. 23 shows a protein identification plan.

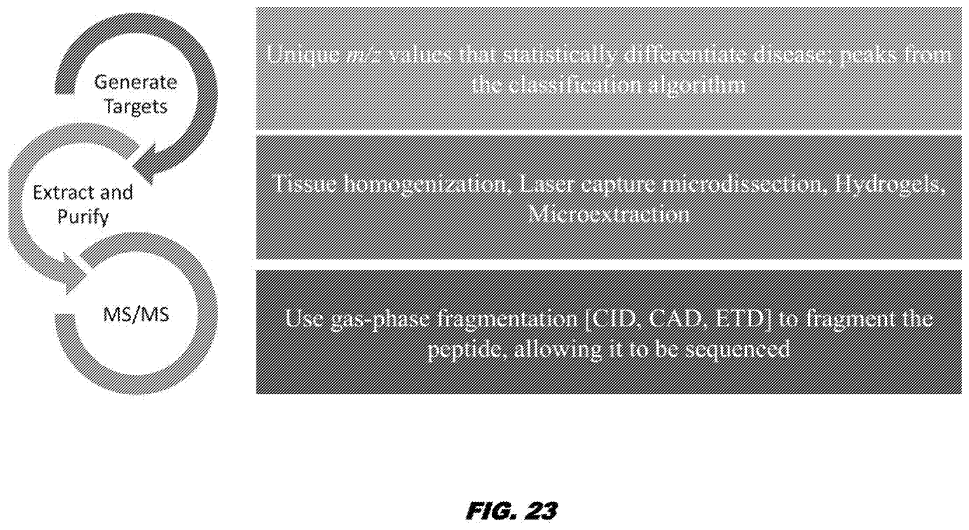

FIG. 24 shows a graph of an unsupervised analysis: lesions in molecular space. Point=one mass spectrum. Structure of data shows separation of classes. Can further probe this molecular space to build a classifier capable of sorting new data.

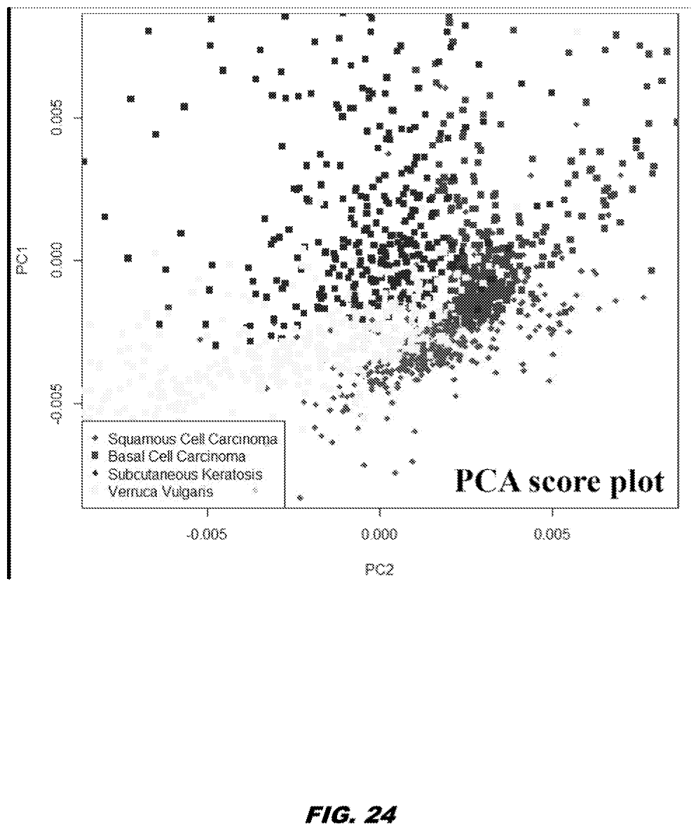

FIG. 25 shows results from a supervised analysis, allowing for pursuit of a molecular diagnosis.

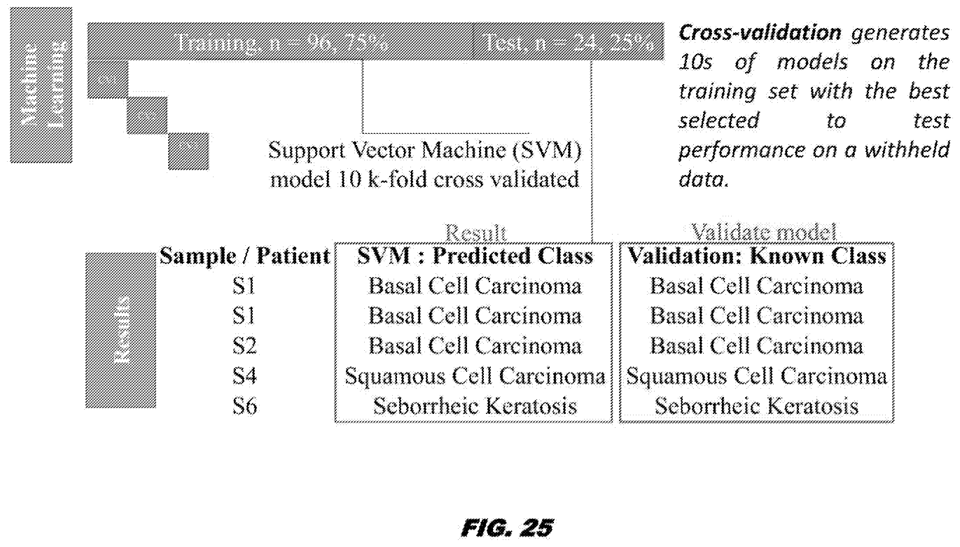

FIG. 26 shows H&E sections for Verruca Vulgaris (left panels), squamous cell carcinoma (middle panels), and Seborrheic Keratosis (right panels). Basal cell carcinoma is noted in blue while squamous cell carcinoma is noted in yellow. Each spot is 300 μm.

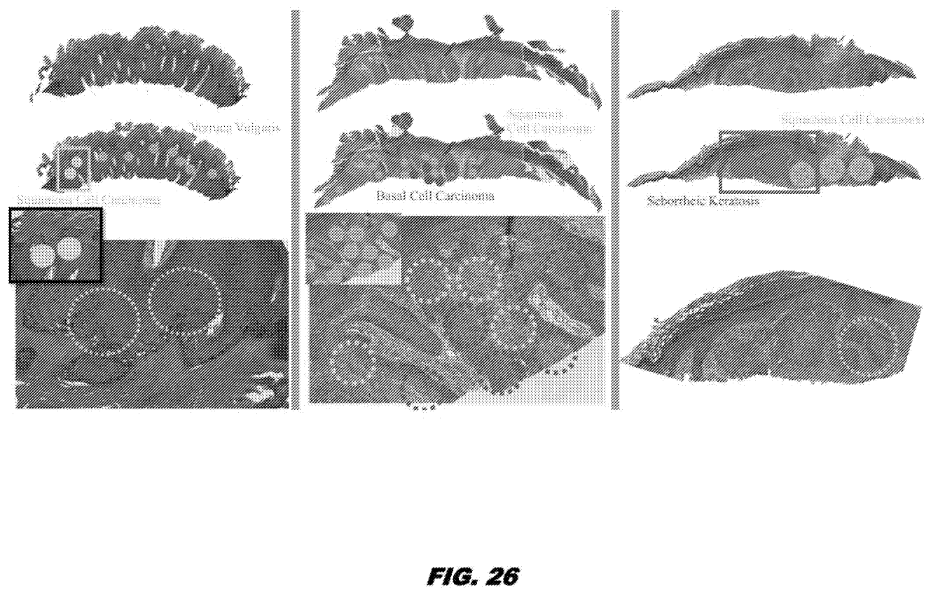

FIG. 27 shows H&E sections for Verruca Vulgaris (top left panel), squamous cell carcinoma (bottom left panel), Basal cell carcinoma (top right panel), and Seborrheic Keratosis (bottom right panel). Each spot is 300 μm.

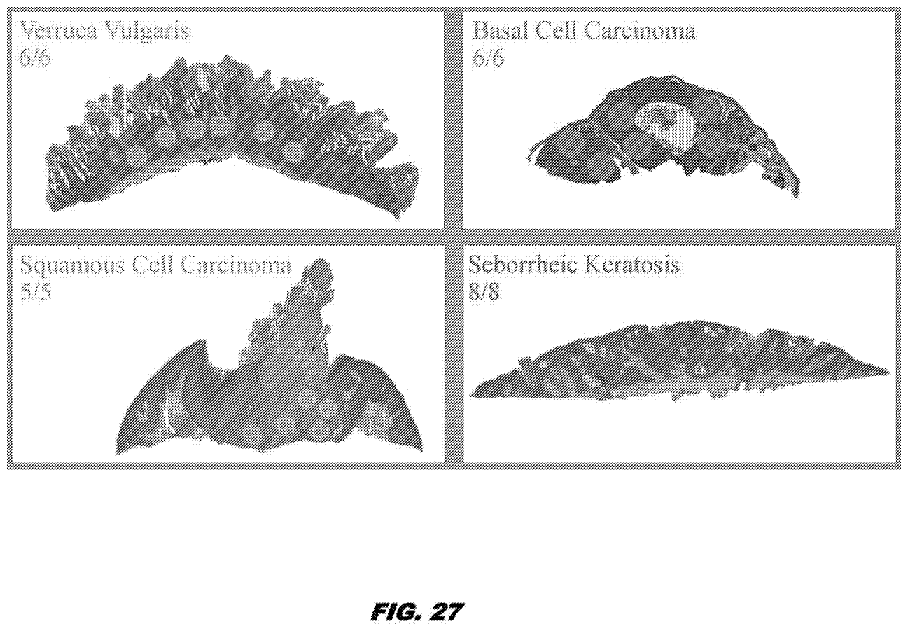

FIG. 28 shows H&E sections for Verruca Vulgaris (top left panel), squamous cell carcinoma (bottom left panel), Basal cell carcinoma (top right panel), and Seborrheic Keratosis (bottom right panel). Each spot is 300 μm.

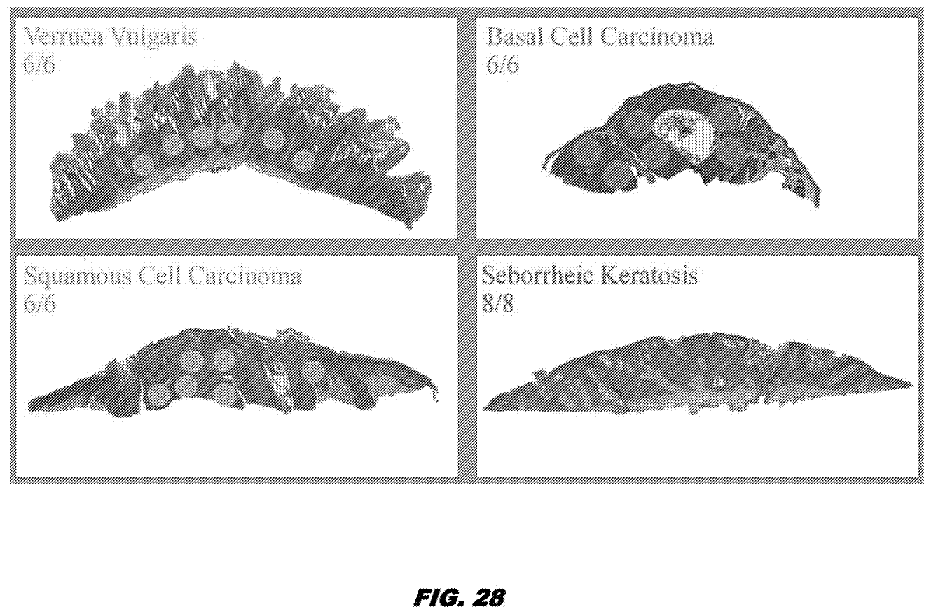

FIG. 29 shows H&E sections for Seborrheic Keratosis classified as basal cell carcinoma. Each spot is 300 μm.

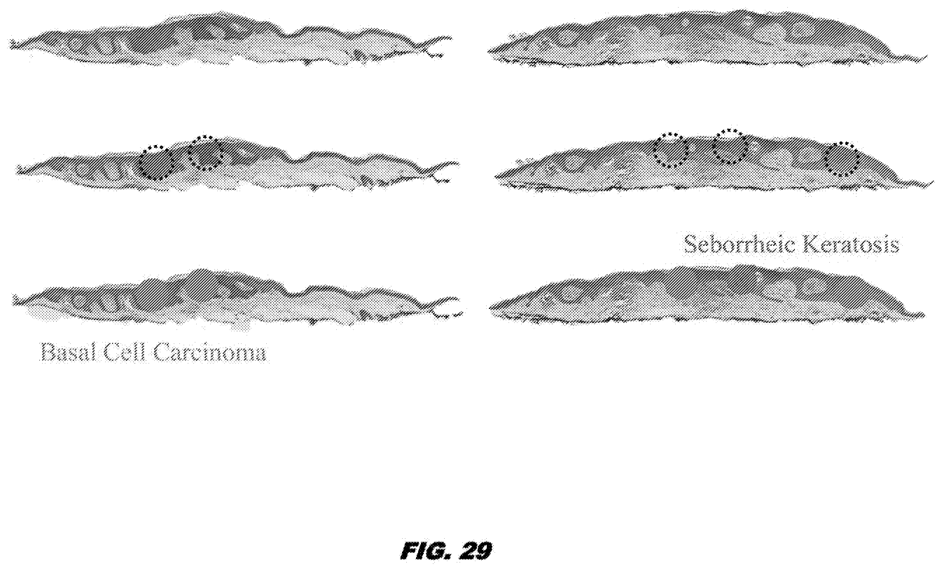

FIG. 30 shows H&E sections for Seborrheic Keratosis classified as basal cell carcinoma. Each spot is 300 μm.

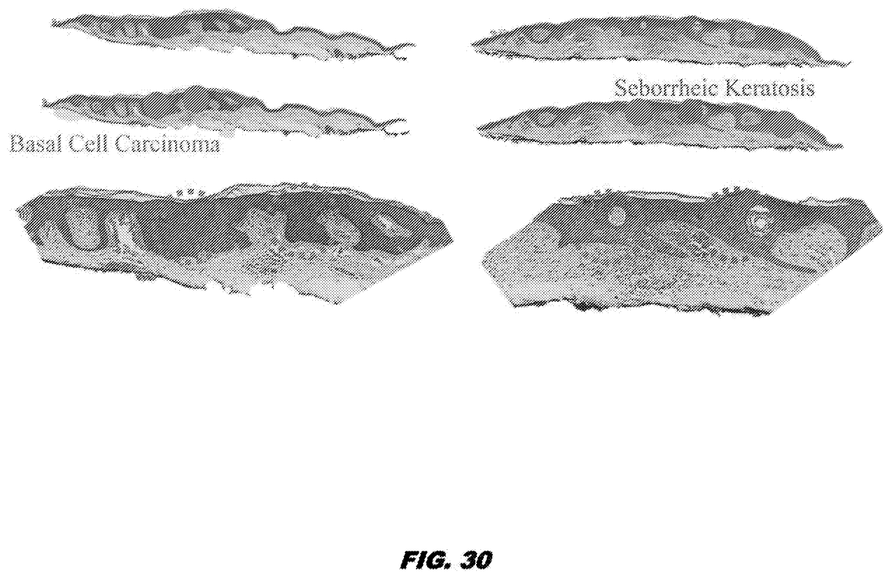

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Carcinoma

Carcinoma is a type of cancer that arises from cells that comprise the skin or the tissue lining organs, such as the liver or kidneys. Some common types of carcinoma include, but are not limited to, basal cell carcinoma, squamous cell carcinoma, renal cell carcinoma, ductal carcinoma in situ, invasive ductal carcinoma, and adenocarcinoma.

### Autoimmune Disorder

An autoimmune disorder is a condition wherein an immune response is mounted against the subject's own cells resulting in the subject's immune system attacking its very own tissue. Non-limiting examples of an autoimmune disorder include psoriasis, psoriatic arthritis, Crohn's disease, rheumatoid arthritis.

### Protein-Based Detection—Mass Spectrometry

By exploiting the intrinsic properties of mass and charge, mass spectrometry (MS) can be resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles that are useful for grading carcinomas and predicting carcinoma patient survival, without regard for the identity of specific proteins.

### Electrospray Ionisation

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (106 to 107 V/m) at the capillary tip. A sample liquid carrying the analyte to be analyzed by the mass spectrometer is delivered to tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258, which are incorporated herein by reference in their entireties.

### Electrospray Ionisation Tandem Mass Spectrometry (ESI/MS/MS)

In ESI tandem mass spectrometry (ESI/MS/MS), one can simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

### Secondary Ion Mass Spectrometry (SIMS)

Secondary ion mass spectrometry, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analyzed by the mass spectrometer in this method.

### LD-MS and LDLPMS

Laser desorption mass spectrometry (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectrometry). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and the separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

### MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms in order to characterize endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a useful quantitative tool. MALDI-TOF-MS also allows non-volatile and thermally labile molecules to be analyzed with relative ease. Without being bound by theory, MALDI-TOF-MS can be useful for quantitative analysis in clinical settings, for toxicological screenings, as well as for environmental analysis. In addition, the application of MALDI-TOF-MS to the quantification of peptides and proteins is also useful. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which can contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al, 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

### Immunohistochemistry

Antibodies can be used in conjunction with both fresh-frozen and/or formalin-fixed, paraffin-embedded tissue blocks prepared for study by immunohistochemistry (IHC). The method of preparing tissue blocks from these specimens has been successfully used in previous IHC studies of various prognostic factors, and/or is well known to those of skill in the art (Brown et al., 1990; Abbondanzo et al., 1999; Allred et al., 1990).

The present invention can also employ immunohistochemistry. This approach uses antibodies to detect and quantify antigens in intact tissue samples. Thin sections of tissue specimens are collected onto microscope slides. Samples that have been formalin-fixed and paraffin embedded must be subjected to deparaffinization and antigen retrieval protocols prior to incubation with an antibody against the target protein of interest. Deparaffinization is accomplished by incubating the slides in xylene to remove the paraffin followed by graded ethanol and water to rehydrate the sections. Antigen retrieval is carried out through incubating the sections in buffer such as tris or citrate with heat which may be introduced via a pressure cooker or a microwave. Sections can then be stained with antibodies using a direct or indirect method.

The direct method is a one-step staining method and involves a labeled antibody (e.g. FITC-conjugated antiserum) reacting directly with the antigen in tissue sections. While this technique utilizes only one antibody and therefore is simple and rapid, the sensitivity is lower due to little signal amplification, such as with indirect methods, and is less commonly used than indirect methods.

The indirect method involves an unlabeled primary antibody (first layer) that binds to the target antigen in the tissue and a labeled secondary antibody (second layer) that reacts with the primary antibody. As mentioned above, the secondary antibody must be raised against the IgG of the animal species in which the primary antibody has been raised. This method is more sensitive than direct detection strategies because of signal amplification due to the binding of several secondary antibodies to each primary antibody if the secondary antibody is conjugated to the fluorescent or enzyme reporter.

### Mass Spectrometry Target Proteins

The present invention provides a protein-based classification of carcinomas. This classification is based on the identification of peaks for at least two peptides, the expression of which correlates with various disease states.

### Mass Spectrometry Profile

In one embodiment, the invention provides for examination of mass spectrometry profiles of proteins from various regions of a skin sample. The sample contains both melanocytic and stromal components, and one can examine either or both of these regions.

The classification model as described herein is based on a peptide signature comprising a number of peaks.

### Classification Model

Spectral classification is achieved using any of various software or processes known by those of ordinary skill in the art. In one exemplary embodiment, spectral classification is achieved by using R language supplied by the GNU project (available from the Free Software Foundation, Boston, Mass.). In alternative embodiment, spectral classification is achieved by using the ClinProTools statistics package supplied by Bruker Daltonics Inc. (Billerica, Mass., USA). In embodiments, spectra are organized and grouped according to the patient sample from which they originate. All spectra belonging to the same diagnosis are loaded into the software as a class with 2 or more classes being loaded for one analysis. All spectra are subjected to preprocessing which includes baseline subtraction, noise level estimation, and normalization to total ion current. Peak boundaries for integration and analysis are manually determined by selection of the monoisotopic peak or automatically by selecting signals with signal-to-noise greater than 3. The peak data can then be used to create a classification model. In embodiments, the peak data are used to create a classification model using an iterative cross-validation approach where the underlying model algorithm is empirically selected based on performance. Before model training under one embodiment, the data undergoes a 70/30 training/test split. 70% of the data is sent for training using a cross-validation approach. The remaining 30% is reserved to test the final model after cross-validation. The cross-validation approach further splits the whole of the training data (70% of the total data) into 70% training and 30% cross-validation. The 70% portion of the data is trained, and tested on the remaining 30%. This process is repeated with different random subsets of the data. The final model is chosen based on accuracy performance.

In an embodiment, the classification model is created using a genetic algorithm. Under one embodiment, a set of peaks are chosen and evaluated for their ability to classify spectra into their correct diagnosis. This set of peaks can then then be crossed with another set of peaks, similar to genetic reproduction and the offspring can be evaluated for their classification ability. Those sets that perform better than the parents can be further crossed with other sets to determine the most optimal set of peaks while those that perform worse, can be discarded. This crossing and evaluation can be carried out over 50 generations to determine the best optimized set of peaks for diagnostic classification. In certain embodiments, the maximum number of peaks to be used is set to 15, but in some embodiments the software can determine the optimal number to include in the model. The maximum number of peaks can be, for example, up to 50. In other embodiments, a maximum of 20 peaks are used. The number of peaks to be used can be, for example, 1, 2, 3, 4, 5, 6, 7, 8, 9, 10, 11, 12, 13, 14, or 15.

Once a model has been optimized, it can be evaluated through cross-validation. One embodiment uses a leave-20%-out crossvalidation approach. In this embodiment, a subset of 20% of the data can be randomly selected to be left out and the remaining 80% can be used to build the classification model. The model can then be applied to the 20% that were originally left out and the accuracy of the classification can be determined. This can be carried out over 10 iterations with a different random 20% left out each time.

Once a model has been optimized through cross-validation, it can be evaluated using the withheld test set. In an exemplary model building phase, the test set data is randomly selected from the annotated data with constraints to avoid any imbalance in the number of data points from each sample group. The model then classifies the test set, and the accuracy of the model is determined by finding the number of true positives and negatives versus false positives and negatives.

Once an optimized classification model has been established, it can be applied to new data in a validation mode, a classification mode, or a combination of thereof In the validation mode, data are organized and identified as to the group to which they belong. The software then classifies the data and evaluates the accuracy of the classification reporting percentages of spectra correctly classified.

The final classification model can be applied to new, unknown data in a blinded fashion. In this classification mode, the researcher and the software are blinded as to the diagnoses of the sample from which the data originated. The software classifies the data into the group that it best matches and reports a list of classification results for each spectrum. Someone with knowledge of the clinical diagnosis of the samples must then evaluate the classification results as compared to the known diagnosis.

### Carcinoma Therapies

Based on the stage of the cancer and other factors, treatment options comprise surgery, immunotherapy, targeted therapy, chemotherapy, or radiation therapy.

Generally, early stage cancer can be treated with surgery alone, but more advanced cancers often require other treatments, including multiple treatments such as adjuvant therapy.

Non-limiting examples of immunotherapies comprise interferon, interleukin-2, pembrolizumab, nivolumab, ipilimumab.

Non-limiting examples of targeted therapies comprise vemurafenib, dabrafenib, trametrinib, and codimetinib, imatinib, and nilotinib.

Non-limiting examples of chemotherapies comprise dacarbazine and temozolomide.

### Pharmaceutical Formulations and Routes of Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The phrase “pharmaceutically or pharmacologically acceptable” can refer to molecular entities and compositions that do not produce adverse, allergic, or other untoward reactions when administered to an animal or a human. As used herein, “pharmaceutically acceptable carrier” includes, for example, any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions for treatment of a subject in need according to the present invention will be via any common route so long as the target tissue is available via that route. This includes intradermal, subcutaneous, intramuscular, intraperitoneal, or intravenous injection. In particular, intratumoral routes and sites local and regional to tumors are contemplated. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients that may include water, binders, abrasives, flavoring agents, foaming agents, and humectants.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

MALDI Imaging Mass Spectrometry Differentiates Squamous Cell Carcinomas, Basal Cell Carcinomas, Verrucas, and Seborrheic Keratoses

Cutaneous squamous lesions can be difficult to distinguish from squamous cell carcinoma, irritated verruca, irritated seborrheic keratosis, or an irritated, squamatized basal cell carcinoma. Reactive atypia or sampling issues can also present problems.

MALDI IMS is a powerful new technology for differentiating Squamous Cell Carcinomas, Basal Cell Carcinomas, Verrucae, and Seborrheic Keratoses.

Our technique unbiasedly differentiated the 4 lesion types with 96% accuracy.

The algorithm developed and employed herein can further incorporate known clinical outcomes to assist in distinguishing borderline or ambiguous lesions. The inventors will characterize the peptides with substantial implication in lesion typing.

MS preprocessing: A large portion of the MALDI-MS data is unspecific noise which can influence the quality of the mathematical model (i.e. a model fits to the noise and not the important molecular data). To address this reality, in certain embodiments, only the peaks are selected from the data. In these embodiments, the dimensionality of the data is reduced (10s of thousands of features (m/zs)) to 100s of features which are much more compatible with machine learning. After extraction of the features (molecular peaks), a table of class vs peaks can be built. For each class there can be multiple observations, and for each observation, there can be hundreds of features.

Machine Learning: In the Example 1 study, once we acquired the totality of the data, we randomly split it into a training and a test set. The training set is comprised of 75% of the samples, while the test set is comprised of 25% of the samples. During training, the test set is COMPLETELY WITHHELD from the training set to prevent biasing our classifier to the data with which we will test it. For training, we cross validate on the training set by randomly subset out 1/10 of the data, training a model, and iteratively repeat this process 10 times (10× k-fold cross validation). From this procedure, we generate 100s of models and we select the one with the best self-cognition accuracy to apply to the test set. Since all classifications are known, we can determine the accuracy of our model on the test set and get a measure of how well it generalizes (i.e. how well it will perform in real world setting), although at a limited scale. The results are a table of unknown data and its classification using our model.

### Example 2

Differentiating Mycosis Fungoides from Psoriasis: A MALDI Imaging Mass Spectrometry Approach

Mycosis fungoides is a common form of cutaneous T-cell lymphoma. Traditionally difficult to diagnose, there is a great need for diagnostics to aid in differentiating from other diseases like psoriasis. Imaging mass spectrometry (IMS) is an analytical tool that provides molecular information from spatially defined regions within FFPE tissues. In dermatopathology, it has been successfully used to differentiate melanoma from melanocytic nevi. Here, we apply this new technology to differentiate mycosis fungoides from psoriasis. In this study, 20 patient samples of either mycosis fungoides (10) or psoriasis (10) were compared. 55 Spectra were collected from psoriasis and 59 from mycosis fungoides samples. The method had an overall classification accuracy of 92.2%, with 94.6% accuracy in determining psoriasis and an 89.8% accuracy in determining mycosis fungoides.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of distinguishing a squamous lesion, the method comprising:
subjecting a sample from a subject to mass spectrometry;
obtaining a sample mass spectrometric profile from the sample;
comparing the sample mass spectrometric profile to a profile obtained from a known normal, a tissue abnormality, a carcinoma sample, or a combination thereof and
identifying the lesion as a carcinoma or tissue abnormality based on the comparison between the sample mass spectrometric profile and the known profile, the tissue abnormality profile, the carcinoma sample profile, or a combination thereof.

2. A method of identifying carcinoma or a tissue abnormality, the method comprising:
subjecting a sample from a subject to mass spectrometry;
obtaining a sample mass spectrometric profile from the sample;
comparing the sample mass spectrometric profile to a profile obtained from a known normal, a tissue abnormality, a carcinoma sample, or a combination thereof; and
identifying the lesion as a carcinoma or tissue abnormality based on the comparison between the sample mass spectrometric profile and the known profile, the tissue abnormality profile, the carcinoma sample profile, or a combination thereof.

3. The method of claim 1 or claim 2, wherein the sample is a skin lesion sample, gastrointestinal lesion sample, a muscle lesion sample, or a bone lesion sample.

4. The method of claim 3, wherein the sample comprises melanocytic components, stromal components, or a combination thereof.

5. The method of claim 1 or 2, wherein the tissue abnormality is Seborrheic Keratosis or Verruca Vulgaris.

6. The method of claim 1 or 2, wherein the carcinoma is basal cell carcinoma, squamous cell carcinoma, renal cell carcinoma, ductal carcinoma in situ, invasive ductal carcinoma, or adenocarcinoma.

7. The method of claim 1 or 2, wherein the tissue abnormality manifests as a result of an autoimmune disorder.

8. The method of claim 7, wherein the autoimmune disorder comprises psoriasis, psoriatic arthritis, Crohn's disease, rheumatoid arthritis, or a combination thereof.

9. The method of claim 1 or 2, wherein one or more peaks from the sample mass spectrometric profile are compared to one or more peaks of the profile obtained from the known normal, the tissue abnormality, the carcinoma sample, or the combination thereof.

10. The method of claim 9, wherein up to twenty peaks from the sample mass spectrometric profile are compared to up to twenty peaks of the profile obtained from the known, normal, the tissue abnormality, the carcinoma sample, or the combination thereof.

11. The method of claim 1 or 2, wherein the mass spectrometric profiles comprise a plurality of molecules.

12. The method of claim 11, wherein the molecules comprise at least one protein, at least one peptide, at least one lipid, at least one metabolite, or a combination thereof.

13. The method of claim 1 or 2, wherein mass spectrometry comprises secondary ion mass spectrometry, laser desorption mass spectrometry, matrix assisted laser desorption/ionization mass spectrometry, electrospray mass spectrometry, or desorption electrospray ionization.

14. The method of claim 1 or 2, further comprising
obtaining or having obtained the sample from the subject.

15. The method of claim 1 or 2 further comprising
performing or having performed histologic analysis on the sample.

16. The method of claim 15, further comprising:
identifying or having identified one or more regions of interest from the histological analysis, wherein the mass spectrometric profile is obtained from one or more regions of interest.

17. The method of claim 1 or 2 further comprising determining or having determined the subject's survival based on the identification.

18. The method of claim 1 or 2 further comprising selecting an effective anti-cancer agent.

19. The method of claim 1 or 2 further comprising administering or having administered to the subject an effective amount of an anti-cancer agent.

20. The method of claim 19, wherein the anti-cancer agent comprises chemotherapy, immunotherapy, toxin therapy, targeted therapy, radiotherapy, or a combination thereof.

21. The method of claim 20, wherein immunotherapy comprises interferon, interleukin-2, pembrolizumab, nivolumab, ipilimumab.

22. The method of claim 20, wherein targeted therapy comprises vemurafenib, dabrafenib, trametrinib, and codimetinib, imatinib, and nilotinib.

23. The method of claim 20, wherein chemotherapy comprises dacarbazine, temozolomide, or a combination thereof.

24. A method of distinguishing a lesion the method comprising:
subjecting a sample from a subject to mass spectrometry;
obtaining a sample mass spectrometric profile from the sample;
comparing the sample mass spectrometric profile to a profile obtained from a known normal, a tissue abnormality, a t-cell lymphoma sample, or a combination thereof; and
identifying the lesion as a t-cell lymphoma or tissue abnormality based on the comparison between the sample mass spectrometric profile and the known profile, the tissue abnormality profile, the t-cell lymphoma sample profile, or a combination thereof.

25. The method of claim 24, wherein the tissue abnormality comprises psoriasis or eczema.

26. The method of claim 24, wherein the t-cell lymphoma comprises mycosis fungoides, Sézary syndrome, primary cutaneous anaplastic large cell lymphoma, lymphomatoid papulosis, granulomatous slack skin disease, pagetoid reticulosis, subcutaneous panniculitis-like T-cell lymphoma, or a combination thereof.

27. A method of screening for the presence of a carcinoma molecular signature in a subject at risk for a carcinoma, the method comprising:
obtaining or having obtained a sample mass spectrometric profile of a tissue sample from the subject;
comparing or having compared the sample mass spectrometric profile to a known normal molecular signature, a tissue abnormality molecular signature, the carcinoma molecular signature, or a combination thereof; and
identifying or having identified the presence of the carcinoma molecular signature in the sample mass spectrometric profile if the sample mass spectrometric profile comprises a molecular signature that is more similar to the carcinoma molecular signature than the normal molecular signature, the tissue abnormality molecular signature, or a combination thereof.

28. The method of claim 27, wherein the carcinoma molecular signature comprises one or a combination of peaks at about m/z 1167.7, 1628.9, 1878.7, or 2207.1.

29. The method of claim 27, further comprising administering to the subject an effective amount of an anti-cancer agent.

30. The method of claim 29, wherein the anti-cancer agent comprises chemotherapy, immunotherapy, toxin therapy, targeted therapy, radiotherapy, or a combination thereof.

31. The method of claim 30, wherein immunotherapy comprises interferon, interleukin-2, pembrolizumab, nivolumab, ipilimumab.

32. The method of claim 30, wherein targeted therapy comprises vemurafenib, dabrafenib, trametrinib, and codimetinib, imatinib, and nilotinib.

33. The method of claim 30, wherein chemotherapy comprises dacarbazine, temozolomide, or a combination thereof.

34. The method of claim 27, wherein the tissue abnormality is Seborrheic Keratosis or Verruca Vulgaris.

35. The method of claim 27, wherein the carcinoma is basal cell carcinoma, squamous cell carcinoma, renal cell carcinoma, ductal carcinoma in situ, invasive ductal carcinoma, or adenocarcinoma.

36. The method of claim 27, wherein the tissue abnormality manifests as a result of an autoimmune disorder.

37. The method of claim 36, wherein the autoimmune disorder comprises psoriasis, psoriatic arthritis, Crohn's disease, rheumatoid arthritis, or a combination thereof.

38. The method of claim 27, wherein the mass spectrometric profile comprises a plurality of molecules.

39. The method of claim 38, wherein the molecules comprise at least one protein, at least one peptide, at least one lipid, at least one metabolite, or a combination thereof.

40. The method of claim 27, wherein the sample mass spectrometric profile is obtained via secondary ion mass spectrometry, laser desorption mass spectrometry, matrix assisted laser desorption/ionization mass spectrometry, electrospray mass spectrometry, or desorption electrospray ionization.

41. The method of claim 27, further comprising
obtaining or having obtained the sample from the subject.

42. The method of claim 27 further comprising
performing or having performed histologic analysis on the sample.

43. The method of claim 42, further comprising:
identifying or having identified one or more regions of interest from the histological analysis, wherein the sample mass spectrometric profile is obtained from one or more regions of interest.

44. The method of claim 27 further comprising determining or having determined the subject's survival based on the identification.
